(19) United States
(12) Patent Application Publication
(10) Pub. No.: US 2005/0203353 A1
(43) Pub. Date: Sep. 15, 2005

(54) Multiple purpose, portable apparatus for measurement, analysis and diagnosis

(76) Inventors: Jie Ma, Birmingham, AL (US); Zuomin Dong, Victoria (CA)

Correspondence Address: Jie Ma, 2628 Crossgate Lane, Birmingham, AL, 35216, US

(21) Appl. No.: 11/068,556

(22) Filed: Feb. 28, 2005

(30) Foreign Application Priority Data
Mar. 10, 2004 (CN) 200410006491.7

(51) Int. Cl. C12M 1/34; A61B 5/00

(52) U.S. Cl. 600/315; 435/287.2

(57) ABSTRACT

The present invention pertains to a portable apparatus for quantitatively measuring the concentration of specific substances in test samples of a lateral flow or microplate assay in medical, biomedical and chemical applications, and for making subsequent analysis and diagnosis. The portable apparatus includes a sample tray for carrying and aligning the test sample in the apparatus; a enclosure that may also serves as the frame of the apparatus; a digital image acquisition system that is used to obtain the digital image of the test sample on the sample tray; and a data display, processing, and analysis unit that is a general purpose or dedicated computer, such as a handheld computer (HHC), a pocket personal computer (PPC), a personal digital assistant (PDA), a palm-top computer, a laptop computer, or a dedicated microprocessor and associated hardware, for measuring the concentration of specific substances in the test sample, and making subsequent analysis and diagnosis, based on the measurement, statistical data, prior knowledge and mathematical model. The stated enclosure and frame, the digital image acquisition system, and the data display, processing and analysis unit are integrated to form the portable apparatus for various applications. The integrated apparatus of this invention, with a possible name—Portable Intelligent Multi-Diagnoser (PIMD), thus forms a portable and multiple-purpose tool for measuring the concentration of specific substances in test samples, and making subsequent analysis and diagnosis in a variety of settings, such as a mobile site, point of care or near patient care, and small laboratories.

### RELATED PRIOR CHINESE APPLICATION

This application claims priority from Chinese Application Number 200410006491.7 filed on Mar. 10, 2004.

### FIELD OF THE INVENTION

The present invention pertains to a portable apparatus and method for quantitatively measuring the concentration of specific substances in test samples of a lateral flow or microplate assay, and making subsequent analysis and diagnosis. More specifically, the invention integrates the measurement on the amount of an analyte present in a subject sample, the analysis and diagnosis by associating this measurement with statistical data, prior knowledge, and mathematical models, as well as the creation and exchange of relevant database. Moreover, this portable apparatus that can be used for both mobile and stationary applications is made by integrating a small general purposes or dedicated computer, imaging system and software.

### BACKGROUND ART OF THE INVENTION

Medical, biomedical and chemical assays can be carried out using various methods and apparatuses. Normally accurate and quantitative results can be only be obtained using complex laboratory-based techniques and apparatuses, and interpreted by professionals with the help of special software. While most simple, manually operated assays, although low in cost and ease of use, can only provide qualitative results. Good examples are the complex medical lab-based, quantitative blood test for cholesterol levels, and the simple user-operated, qualitative home pregnant test.

Medical and biomedical tests are traditionally carried out using specialized and expensive biomedical and chemical systems in hospitals and laboratories, through a number of experiments, comparing tests, and interpretations by specialists. The introduction of Enzyme-linked Immunosorbent Assay (ELISA) has considerably improved some of these tests using bench top equipments. The ELISA laboratory technique consists of three major components: ELISA immunological and biochemical reaction, ELISA reader, and data analysis software. The ELISA reaction presents antigen or antibody concentrations through the change of color and/or intensity of light in multiple microplates. These results are then collected and interpreted by the ELISA reader using fluorescence and absorbance measurements. Different light sources and filters with different bandpasses are used to facilitate the measurement of a specific antigen or antibody concentration more effectively. Using an internal, standard calibration curve, the measured light intensity is translated into corresponding antigen or antibody concentration. Dedicated analysis software compares the antigen or antibody concentration with the median value of the population from statistics and incorporates other influencing factors to produce the corresponding diagnosis for the application. A typical ELISA reader is the Synergy HT Multi-Detection Microplate Reader from Bio-Tek Instruments Inc., which utilizes a dual-optics design combined with monochromator wavelength selection with absorbance, fluorescence and luminescence reading modes (http://www.biotek.com/products/detection.php, 2003).

Although these recent developments are encouraging, the bench-top ELISA technology remains the domain of specialized laboratories in which sample preparation, instrumentation and result analysis are carried out. The required laboratory setting and demanding multiple step/operator process prevents its widespread use, and greatly mitigate the benefits of the technology.

Today's rapid pace of life requires quick analysis and diagnosis ranges from risk assessments of prenatal Down syndrome and neural tube defect fetus screening, cardiovascular disease and prostate cancer, to the monitoring of food safety, environment conditions, and illegal replica detection (through test on the quantity of key ingredients of various food, soft-drink, medicine, health, paper and chemical products). These analysis and diagnosis are to be carried out using hand-held devices enabling doctors to rapidly obtain lab-quality diagnosis at the patient bedside, or portable detectors to warn inspectors of infection, contamination or poor replica at various sites, or easy to use, health monitoring devices for home use by non-professionals.

### SUMMARY OF THE INVENTION

This invention provides a quick, multiple functional, and on-site measurement, analysis and diagnosis method and apparatus that satisfies these stated needs.

The technology pertained in this invention offers many advantages over traditional large, cumbersome and labor intensive laboratory experiments in terms of speed, cost, chemical usage, contamination, efficiency, safety and automation.

The invented technique and apparatus combine the immunology, biochemical, or chemistry reaction, the reader, and the data analysis software of the ELISA equivalent into one portable and self-contained, palm, laptop or desktop device. The invention merges the traditionally separated testing and diagnosis functions into one stand-alone, cost-effective, portable, and fully-integrated device.

Different from other dedicated, portable assay result readers, such as the lateral flow assay apparatus by Plito, et al, which uses specially designed hardware to carry out a specific immunology assay, as disclosed in U.S. Pat. No. 6,136,610, (issued 24 Oct. 2000 to Plito, et al), this invention used a general purpose small computer or microprocessor, an imaging system, and software as the core of the apparatus. The small computer and microprocessor of various types can also be referred as a generalized data display, processing, and analysis unit.

This design allows different assay calibration, result analysis, diagnosis, and communication software to be installed onto the apparatus to carry out a variety of medical, biomedical and chemical assays through the quantitative measurement on the concentration of specific substances in test samples of a lateral flow or microplate assay. In addition, the computing capability of the data display, processing, and analysis unit allows the assay calibration, result analysis, diagnosis, as well as data acquisition and communication to be carried out using one apparatus. In particular, quick, on-site diagnosis can be made based on the measured substance concentration in the test sample and the analysis using statistical data, prior knowledge, and mathematical models stored on the computer or the data display, processing, and analysis unit. With the use of a general purpose small computer, including handheld computer (HHC), pocket personal computer (PPC), personal digital assistant (PDA), palm-top computer and laptop computer, data communication and program transfer between the portable apparatus and with the central computers, as well as connections to all computer peripherals can be easily accomplished.

In short, the invented apparatus present the following unique features and advantages:
Effective integration of several cutting-edge technologies, including portable HHC, PPC and PDA, digital image acquisition and processing, low-cost microprocessor, internet and wireless data communication, as well as pattern recognition, statistical and task-dedicated analysis software.
Broad applications in medical, biomedical, chemical, and many other areas, as long as light color and intensity change can be observed on a testing stripe, cartridge, or microplate.
Low cost and ultimate flexibility due to the direct use of the fast advancing and mass produced, general purpose small computer hardware and programming tools, or commonly used microprocessors.

As an enabling technology for scientists, medical professionals, and non-professional home users, the invented apparatus has the potential to significantly improve biomedical, life sciences, and chemical analysis and diagnosis by providing a low-cost, versatile, and highly capable on-site tool for measuring specific substance concentration in test samples and for carrying on-site analysis and diagnosis using statistical data, prior knowledge and mathematical models.

An object of the invention is to provide a portable apparatus for measuring specific substance concentration in test samples, and making subsequent analysis and diagnosis in a variety of settings, such as a mobile site, point of care or near patient care, and small laboratories.

Another object of the invention is produce the assay analysis and diagnosis apparatus using a small, general purpose computer or data display, processing and analysis unit, such as a handheld computer (HHC), a pocket personal computer (PPC), a personal digital assistant (PDA), a palm-top computer, or a laptop computer.

Another object of the invention is develop the assay analysis and diagnosis apparatus alternatively using a low-cost microprocessor and associated hardware to serve as the data display, processing and computation unit to further lower the production costs of the apparatus while retaining its versatility and functionality.

Another object of the invention is to provide a method and apparatus that quantitatively measure the concentration of specific substances in test samples of a lateral flow or microplate assay to a high degree of accuracy.

Another object of the invention is an apparatus, by which the measured substance concentration in test sample can be analyzed using stored statistical data, prior knowledge, and mathematical models.

Another object of the invention is an apparatus, by which proper diagnosis on the subject matter can be made based on the quantitative measurement of substance concentration in the test sample and computing analysis of the measured results.

Another object of the invention is an integrated apparatus, in which the quantitative measurement on the concentration of specific substances in test sample of a lateral flow or microplate assay; analysis based on the measured substance concentration, and stored statistical data, and computer models; construction and maintenance of a user/patient and sample database; and communications with the central database, are carried out.

Another object of the invention is to use a digital camera, special illumination, and optical filters to acquire images of different color and light intensity from the sample of a lateral flow or microplate assay, and to map the sensed color and light intensity into corresponding concentration of specific substances in the test sample, based on a stored image light color/intensity to sample substance concentration calibration curve.

Another object of the invention is an integrated apparatus, on which the digital images of the test sample of a lateral flow or microplate assay can be acquired, their known sample substance concentrations can be entered, and the image light color/intensity to sample substance concentration calibration curve can be generated, for this and other apparatuses, and for present and later use.

Another object of the invention is an assay analysis and diagnosis apparatus, made partially using a general purpose small computer, in which the relevant information of the sampled subjects or people can be collected, updated, stored and transferred to the central computers and other apparatuses, to support the construction and maintenance of a computer database with both statistical and individual-specific data.

Another object of the invention is an assay analysis and diagnosis apparatus, made partially using a general purpose small computer, in which subject sample related software, statistical data, users' database, and sample calibration data and/or model can be transferred back and forth between the apparatus and the central computers and/or other apparatuses.

Another object of the invention is an assay analysis and diagnosis apparatus, made partially using a general purpose small computer, in which various computer software and programming environments, such as database programming and management, speed sheets, word processor, file transfer protocols, C, BAISC or other programming tools, can be directly used.

Another object of the invention is an assay analysis and diagnosis apparatus, made partially using a general purpose small computer, in which various computer peripherals, such as printer, network connection, wireless communication, storage devices, can be directly used.

Another object of the invention is to provide a method and apparatus that quantitatively measure the concentration of specific substances in the test sample of a lateral flow or microplate assay by automatically detecting the areas of color and light intensity changes in the sample image, thus incorporating the reactions from the entire active zone and ensuring the accuracy of sample alignment.

Another object of the invention is to provide an apparatus and method that quantitatively measure the concentration of specific substances in the test sample of a lateral flow or microplate assay by automatically using an area of the background and an area of the active zone of the sample as references for interpreting the color and light intensity of the sample image, thus ensuring the measurement accuracy under varying illumination, the back materials of the sample, optical filters, and other changes imposed by environment and production variations.

Another object of the invention is to provide a method and apparatus that quantitatively measure the substance concentrations of multiple test samples of a lateral flow or microplate assay by simultaneously reading and processing several different images on the sample strip and/or sample tray.

Another object of the invention is an assay analysis and diagnosis apparatus, made using a small computer, an image acquisition system, structure illumination, filters and portable power supply, and contained in an enclosure with a removable sample tray that can carry the sample strip(s) and microplate(s).

With these objects in mind, an embodiment of the present invention provides a method for performing a lateral flow assay. The method includes depositing a sample on a test strip or micro disc at an application region, detecting a first detection signal in the form of changing color and light intensity, arising from the test strip in the first detection zone, and generating a baseline for the first measurement zone by interpolating between values of the detection signal outside of the first measurement zone and inside of the first detection zone. The method may include locating a beginning boundary and an ending boundary for the first measurement zone on the test strip. Additional detection zones having measurement zones may also be incorporated with the embodiment.

In summary, the present invention is a portable apparatus, for performing a variety of medical, biomedical and chemical assays, assay based analyses, and diagnosis, comprising
a sample tray that can carry one or multiple test samples for testing;
an enclosure of the apparatus that houses all components and serves as the frame;
an opening on the enclosure to accommodate the sample tray that carries the test sample(s) for acquiring digital image information of the sample;
an imaging acquisition system in the enclosure, which is used for acquiring digital image information of the sample;
a data display, processing, and analysis unit that carries out the measurement, analysis and diagnosis on the test sample, using the acquired image information; the unit can also receive instructions, display and output results.

Among these, the enclosure and frame of the apparatus with the sample tray inserted at least contains the image acquisition system to ensure that the sample tray and the image acquisition system have a fixed relative position inside the enclosure. The image acquisition system is connected to the data display, processing, and analysis unit through digital interface inside of the enclosure, thus forming a portable device for various applications.

The stated portable apparatus of this invention, wherein the data display, processing, and analysis unit for carrying out measurement, analysis, and diagnosis is a general purpose computer that fits within the enclosure of the apparatus, such as a handheld computer (HHC), a pocket personal computer (PPC), a personal digital assistant (PDA), a palm-top computer, or a laptop computer.

The stated portable apparatus of this invention, wherein the data display, processing, and analysis unit for carrying out measurement, analysis, and diagnosis is a general purpose computer, such as a handheld computer (HHC), a pocket personal computer (PPC), a personal digital assistant (PDA), a palm-top computer, or a laptop computer, which resides outside of the apparatus. This computer can be connected and disconnected to the apparatus through a standard interface mounted on the enclosure of the apparatus.

The stated portable apparatus of this invention, wherein the data display, processing, and analysis unit is a dedicated component that fits within the enclosure of the apparatus, which include a microprocessor and associated hardware. This dedicated component is connected to the image acquisition system of the apparatus through a standard interface.

The stated portable apparatus of this invention, wherein the data display, processing, and analysis unit is a dedicated component, such as a microprocessor and associated hardware. This dedicated component can be connected and disconnected to the apparatus from outside through a standard interface mounted on the enclosure of the apparatus.

The stated portable apparatus of this invention, wherein the image acquisition system hardware consists of a digital camera or the lens head of a digital camera that can be connected and disconnected to the frame and enclosure of the apparatus. The image acquisition system hardware is connected to the digital interface of the data display, processing, and analysis unit.

The stated portable apparatus of this invention, wherein the image acquisition system hardware consists of a digital camera or the lens head of a digital camera, special illumination, and optical filters, which are all fixed to the frame inside of the enclosure of the apparatus. The image acquisition system hardware is connected to the digital interface of the data display, processing, and analysis unit.

The other aspect of this invention is a portable apparatus, for performing a variety of medical, biomedical and chemical assays, assay based analyses, and diagnosis, comprising
a sample tray that can carry one or multiple test samples for testing;
an enclosure of the apparatus that houses all components and serves as the frame;
an opening on the enclosure to accommodate the sample tray that carries the test sample(s) for acquiring digital image information of the sample;
an imaging acquisition system in the enclosure, which is used for acquiring digital image information of the sample; the imaging acquisition system includes a digital camera or the lens head of a digital camera, special illumination, and optical filters; which are fixed onto the frame of the apparatus;
a data display, processing, and analysis unit that carries out the measurement, analysis and diagnosis on the test sample, using the acquired image information; the unit can also receive instructions, display and output results.

Among these, the enclosure and frame of the apparatus with the sample tray inserted contains the image acquisition system to ensure that the sample tray and the image acquisition system have a fixed relative position inside the enclosure. The image acquisition system and the enclosure and frame of the apparatus form one body. In use, the image acquisition system is connected to the data display, processing, and analysis unit through the digital interface that is mounted on the enclosure of the apparatus, thus forming a portable device for various applications.

The stated portable apparatus of this invention, wherein the data display, processing, and analysis unit is a general purpose computer that can be connected to the frame or enclosure of the apparatus.

The stated portable apparatus of this invention, wherein the general purpose computer can be a handheld computer (HHC), a pocket personal computer (PPC), a personal digital assistant (PDA), a palm-top computer, or a laptop computer.

The stated portable apparatus of this invention, wherein the data display, processing, and analysis unit is a dedicated component that includes microprocessor and associated hardware.

The other aspect of this invention is a portable apparatus, for performing a variety of medical, biomedical and chemical assays, assay based analyses, and diagnosis, comprising
a sample tray that can carry one or multiple test samples for testing;
an enclosure of the apparatus that houses all components and serves as the frame;
an opening on the enclosure to accommodate the sample tray that carries the test sample(s) for acquiring digital image information of the sample;
an imaging acquisition system in the enclosure, which is used for acquiring digital image information of the sample; the imaging acquisition system includes a digital camera or the lens head of a digital camera, special illumination, and optical filters; which are fixed onto the frame of the apparatus;
a data display, processing, and analysis unit that carries out the measurement, analysis and diagnosis on the test sample, using the acquired image information; the unit can also receive instructions, display and output results. This data display, processing, and analysis unit is a dedicated component mounted on the frame of the apparatus, which consists of a general-purpose microprocessor and all associated hardware, including display screen.

Among these, the enclosure and frame of the apparatus with the sample tray inserted contains the image acquisition system and the dedicated component. Within the enclosure, the relative position between the digital camera or the lens head of the digital camera of the image acquisition system and the inserted sample tray is fixed, thus forming a portable device under the enclosure for various applications.

The stated portable apparatus of this invention, wherein the data display, processing, and analysis unit includes means for operation and data entry, including control and navigator buttons, display and touch screen, USB port connector, and PocketPC USB port connection; as well as AC Adapter connector and AC power supply.

The stated portable apparatus of this invention, wherein the data display, processing, and analysis unit includes stylus and on screen keyboard.

All configurations of the stated portable apparatus of this invention, wherein the data display, processing, and analysis unit can directly connect to various computer peripheral devices, including but not limited to the data input and output devices, speaker, microphone, infrared port, CompactFlash card slot, PC card slot, Secure Digital card slot, dock port and portable keyboard. Various wireless connections, including IrDA, GSM/GPRS, CDMA and Bluetooth, may be used.

All configurations of the stated portable apparatus of this invention, wherein the sample cartridge can carry one or more samples of the lateral flow or microplate assay.

All configurations of the stated portable apparatus of this invention, wherein the sample cartridge has a pocket or surface with certain width and length to hold a sample of the lateral flow or microplate assay to observe the color and light intensity of the test sample image and their changes.

All configurations of the stated portable apparatus of this invention, wherein the sample cartridge has multiple pockets or surfaces with certain width and length to hold multiple samples of the lateral flow or microplate assay to observe the color and light intensity of the sample and their changes. These samples may be separated with blank spaces.

All configurations of the stated portable apparatus of this invention, wherein the sample cartridge has a line mark to ensure the position alignments between the sample, the sample cartridge, and the enclosure of the apparatus.

All configurations of the stated portable apparatus of this invention, wherein the sample cartridge has one or multiple pockets or surfaces of circular, square, rectangular and other shapes to hold samples of microplate assay to observe the color and light intensity of the image sample and their changes.

All configurations of the stated portable apparatus of this invention, wherein a transparent cover is placed on top of the sample, or between the illumination light and the sample.

All configurations of the stated portable apparatus of this invention, wherein the image acquisition system also include filters and illumination.

All configurations of the stated portable apparatus of this invention, wherein the image acquisition system for sample strips and/or microplates with a reflective backing comprises a digital camera or the lens head of a digital camera, optical filters, an illumination (or light) source, illumination enhancement and close-up lenses, and test strips or microplates with a reflective backing.

All configurations of the stated portable apparatus of this invention, wherein the image acquisition system for sample strips and/or microplates with a transparent or transparent backing comprises a digital camera or the lens head of a digital camera, optical filters, an illumination (or light) source, illumination enhancement and close-up lenses, and test strips or microplates with a transparent or transparent backing.

All configurations of the stated portable apparatus of this invention, wherein a portable and plug-in power supply, preferably with a voltage stabilizer, and control device provide power to image acquisition system and the data display, processing, and analysis unit.

All configurations of the stated portable apparatus of this invention, wherein a regular or rechargeable battery, preferably with a voltage stabilizer, and control device provide power to image acquisition system and the data display, processing, and analysis unit.

All configurations of the stated portable apparatus of this invention, wherein the data display, processing, and analysis unit reads the color and light intensity of image of the test sample of a lateral flow or microplate assay using the image acquisition system, maps the sensed color and light intensity into the corresponding concentration of specific substances in the test sample, based on a stored light color/intensity to substance concentration calibration curve.

All configurations of the stated portable apparatus of this invention, wherein the measurement and analysis on the concentration of specific substances in the test samples of a lateral flow or microplate assay are based on the statistical data, prior knowledge and computer models of the subject matter, stored in the data display, processing, and analysis unit.

All configurations of the stated portable apparatus of this invention, wherein the analysis on the results of the lateral flow or microplate assay and the diagnosis from the results of the analysis are based on the quantitative measurement on the concentration of specific substances in test samples and computer analysis using the data display, processing, and analysis unit.

All configurations of the stated portable apparatus of this invention, wherein the data display, processing, and analysis unit carries or operates on the programs for acquiring, processing and analyzing digital images, and the programs for calibrating and recording the relations between the light color/intensity of sample image and the concentration of specific substances in the test sample.

All configurations of the stated portable apparatus of this invention, wherein the data display, processing, and analysis unit runs a variety of dedicated application software that are used for building and maintaining the information database of the users and/or patients; carrying out quantitative measurement of specific substances in the test sample; performing analysis based on the measured concentration of the specific substances, statistical data, prior knowledge, and mathematical models; and making diagnosis based on the results of the analysis. This data display, processing, and analysis unit in the portable apparatus also transfer the related information and the results of the analysis and diagnosis to and from the central computer and/or other portable apparatuses.

All configurations of the stated portable apparatus of this invention, wherein the data display, processing, and analysis unit can run a variety of application software that are required for carrying out the medical, biomedical and chemical assays, sample substance concentration measurement, analysis and diagnosis, such as the risk assessments of maternal Down and neural tube defect fetus, cardiovascular disease, and prostate cancer, and the monitoring and inspection of food and environment. These application software tools with user interface implemented in different languages can be quickly loaded on and unloaded off the portable apparatus for different applications.

All configurations of the stated portable apparatus of this invention, wherein the data display, processing, and analysis unit can be used to collect and update the relevant information of the sampled subjects or the users/patients, to store the information in a database, and to transfer the information to the central computers and other portable apparatus, in order to support the construction and maintenance of a computer database with both statistical and individual-specific data, such as user/patient database.

All configurations of the stated portable apparatus of this invention, wherein the data display, processing, and analysis unit can use various software, statistical data, users' database, sample calibration data, and computer models to process the information related to the subject sample; and can transfer these software, information and data back and forth to the central computer and/or other portable apparatuses.

All configurations of the stated portable apparatus of this invention, wherein the data display, processing, and analysis unit can directly use various computer software and programming environments, including but not limited to database programming and management, spread sheets, word processor, file transfer protocols, C, BAISC and other programming tools.

All configurations of the stated portable apparatus of this invention, wherein the data display, processing, and analysis unit can directly use various computer peripherals, including but not limited to printer, network connection, wireless communication, storage devices and attachable keyboard.

All configurations of the stated portable apparatus of this invention, wherein the data display, processing, and analysis unit obtains the color and light intensity of the image of the test sample from the image acquisition system of the apparatus; quantitatively measuring the concentrations of specific substances in the test sample using the stored sample image light color/intensity to sample specific substance concentration calibration curve; and automatically detects the boundaries and area of the interested color and light intensity expressing region on the sample image using image processing techniques, thus accurately detecting the color and light intensity of the interested region on the sample image and eliminating possible misalignment between the sample, sample tray and the frame of the apparatus.

All configurations of the stated portable apparatus of this invention, wherein the data display, processing, and analysis unit quantitatively measures the concentrations of specific substances in the sample using the color and light intensity of the acquired image of the sample, and the stored sample image light color/intensity to sample substance concentration calibration curve. The measurement uses a light color/intensity benchmark spot on the sample tray and an area on the sample adjacent to the active zone as light color and intensity background references, ensuring measurement accuracy under varying illumination, sample back material and optical filters, different apparatuses, and other changes that might be imposed by environment and production variations.

All configurations of the stated portable apparatus of this invention, wherein the data display, processing, and analysis unit in the apparatus quantitatively measures the concentrations of specific substances in multiple test samples of a lateral flow or microplate assay by acquiring one image, dividing the image for each sample, and processing the divided image of each sample using different algorithms.

All configurations of the stated portable apparatus of this invention, wherein the apparatus can be used in a method for performing and interpreting a lateral flow assay. The method includes depositing a sample on a test strip at an application region, detecting a first detection signal, in the form of changing color and/or light intensity, arising from the test strip in the first detection zone, and generating a baseline for the first measurement zone by interpolating between values of the detection signal outside of the first measurement zone and inside of the first detection zone. The method may include locating a beginning boundary and an ending boundary for the first measurement zone on the test strip. Additional detection zones having measurement zones may also be incorporated with the embodiment.

All configurations of the stated portable apparatus of this invention, wherein the apparatus can be used in a method for performing and interpreting a lateral flow assay includes providing a test strip on a cartridge, where the test strip includes a first analyte binding agent coupled to a detection agent and a second analyte binding agent. The method further includes depositing a sample on an application region of the test strip, where at least a portion of the sample binds to the first analyte binding agent coupled to the detection agent to form a first analyte binding agent complex, the first analyte binding agent complex moving by lateral flow to a first detection zone that includes a measurement zone, where at least a portion of the first analyte binding agent complex binds to the second analyte binding agent in the first measurement zone to form a second complex. In addition, the method also includes detecting an intensity of a first detection signal, in the form of changing color and/or light intensity, arising in the first detection zone, generating a baseline of signal intensity from the first measurement zone, and quantifying a value of signal intensity representative of the second complex with respect to the baseline.

All configurations of the stated portable apparatus of this invention, wherein the device can be used for:
the quantitative measurement of the concentration of specific substances in test samples of a lateral flow or microplate assay and analysis;
the construction and maintenance of the sample related information database of the users and/or patients, and the exchange of information with the central database on the host computer;
the quantitative analysis and diagnosis based on the measured substance concentration in the test sample, stored statistical data, prior knowledge, and mathematical models.

### BRIEF DESCRIPTION OF THE DRAWINGS

FIG. 1 is an isometric view showing embodiments of the present invention through a typical implementation.

FIG. 2 is a schematic view of hardware and software components of the present invention.

FIG. 3 is a see-through isometric view showing embodiments of the present invention through a typical implementation.

FIG. 4 is an exploded isometric view showing embodiments of the present invention through a typical implementation.

FIG. 5 is an isometric view showing two embodiments on the arrangements of the structural illumination, filters, a digital camera or the lens head of a digital camera, and the sample tray of the apparatus. An arrangement for processing non-transparent, reflective sample is shown in FIG. 5(a), an arrangement for transparent sample is shown in FIG. 5(b).

FIG. 6 includes pictures that illustrate the front (a) and back (b) of the present invention through a typical implementation.

FIG. 7 illustrates one configuration of the sample cartridge that contains one test strip, a close-up of the sample images, and the operation of the sample carriage through a typical implementation.

FIG. 8 illustrates the arrangements of both a single (FIG. 8(b)) and multiple (FIG. 8(c)) test strips within the sample tray of the present invention through a typical implementation.

FIG. 9 illustrates the arrangements of rectangular and/or circular microplates in a single array (FIG. 9(b)) and multiple array (FIG. 9(c)) arrangements within the sample tray of the present invention through a typical implementation.

FIG. 10 illustrates the operation of the present invention and its graphical user interface through a typical implementation.

FIG. 11 illustrates the user's interface of the onboard program that acquires the digital images of the test samples and their known concentrations of specific substances to generate the image light color/intensity to sample substance concentration calibration curve through a typical implementation in Chinese language.

FIG. 12 illustrates the user interface in Chinese language of the application (or user/patient) database construction and maintenance module of the data acquisition, assay analysis and diagnosis software during the operation of the present invented apparatus through a typical implementation.

FIG. 13 illustrates the sample image display of the data acquisition, assay analysis and diagnosis software during the operation of the invented apparatus through a typical implementation in Chinese language.

FIG. 14 illustrates the peripherals of small computer and their incorporation to the present invention through a typical implementation.

### DETAILED DESCRIPTION OF EMBODIMENTS OF THE INVENTION

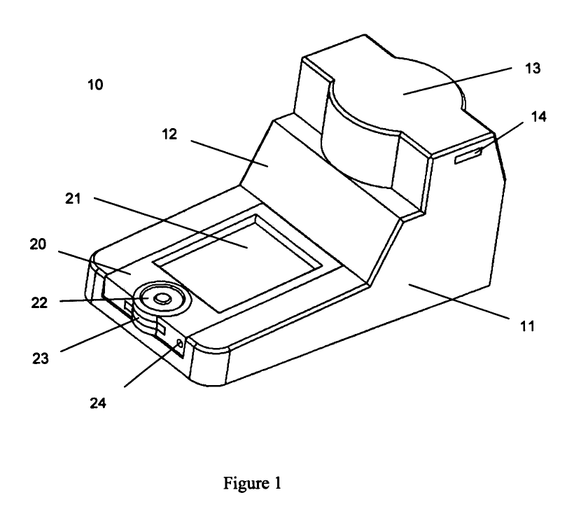

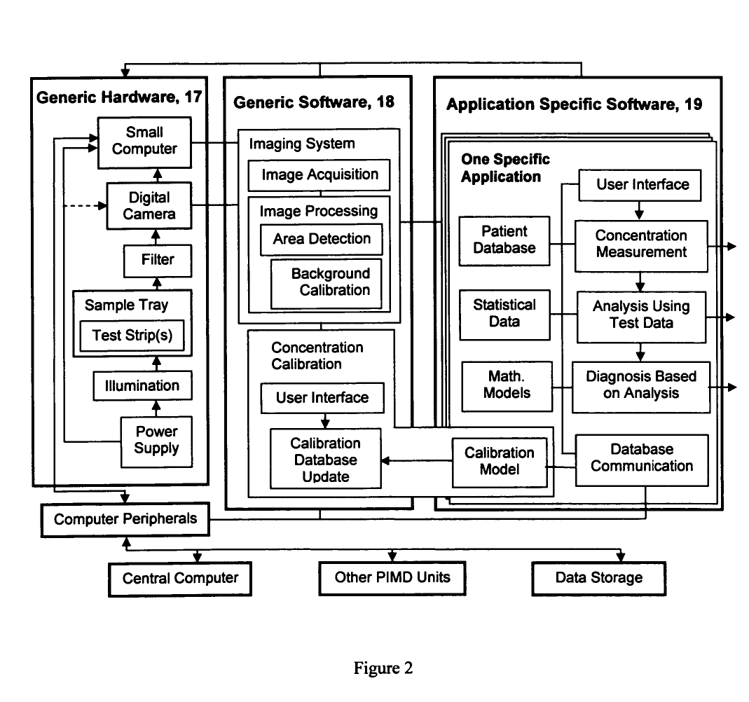

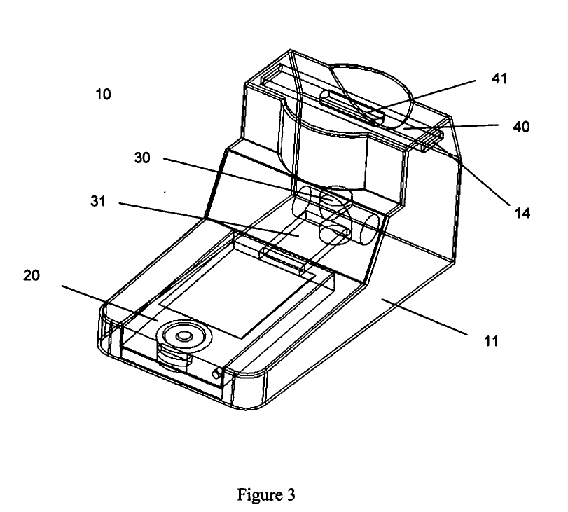

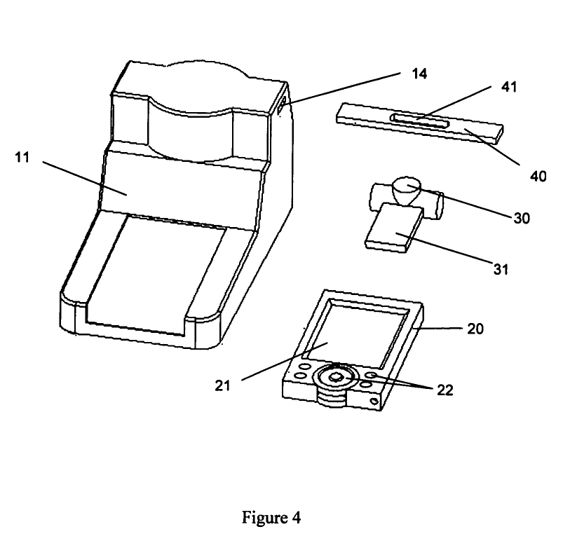

Referring to the drawings, an example implementation of the preferred embodiment of the present invention is shown in an isometric view in FIG. 1. A schematic view of hardware and software components of the invention is given in FIG. 2. Further details of the example apparatus are revealed in the see-through isometric view in FIG. 3 and the exploded isometric view in FIG. 4.

In the embodiment shown in the drawings, the invented apparatus 10 comprises an onboard, general purpose small computer, or data display, processing and analysis unit (a PDA as illustrated) 20, its touch screen 21, navigator and operation buttons 22, USB connector 23 and AC adapter connector 24. Also shown are the enclosure of the invented apparatus 11, an opening 14 on the enclosure to accommodate the sample tray, a sample tray 40, the slot on the sample tray for carrying test sample(s) 41, the imaging system chamber 13 and the digital camera or lens head chamber 12 of the invented apparatus, a digital camera or the lens head of a digital camera 30 and its connector to the small computer, or data display, processing and analysis unit 31 (through a CF card at this instance).

As the data display, processing and analysis unit, the PDA can be replaced by any general purpose small computers, including handheld computer, pocket personal computer, palm-top computer and laptop computer. Alternatively a microprocessor and dedicated hardware may be used to serve as the data display, processing and computation unit to further lower the production costs of the apparatus while retaining its versatility and functionality.

FIG. 2 presents the three major functional components of the invented apparatus, their sub-components, and their functional relations. These include
the hardware 17 of the invented apparatus, consisting of the onboard computer, or data display, processing and analysis unit, the attached digital image acquisition hardware, sample tray, and power supply;
the generic software 18 of the invented apparatus, loaded on the onboard computer, or data display, processing and analysis unit, for supporting the imaging system and for generating the concentration calibration curve;
various application specific software 19 of the invented apparatus, loaded on the onboard computer, or data display, processing and analysis unit, for a variety of applications that require medical, biomedical and chemical assays; and
   Other related functional components, such as computer peripherals and data storage, are also revealed.

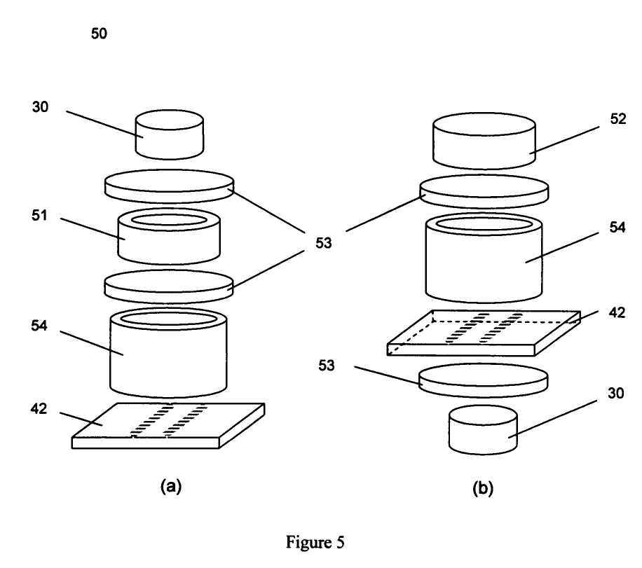

The generic hardware 17 of the invented apparatus supports the computer imaging system by providing sample support, illumination, and image acquisition. Depending upon the types of the sample, two different imaging systems 50 are given in FIGS. 5(a) and 5(b). As the key component of the hardware 17 of the invented apparatus, the onboard small computer, or data display, processing and analysis unit, provides the computing power needed for the image processing, sample analysis, pattern recognition, and diagnosis. It also serves as the control computer to maintain various databases as well as the communications with other computers and units of this invention.

The generic software 18 of the invented apparatus is a collection of application independent, onboard computer programs for acquiring and processing the sample image, identifying the location of the interested color/intensity image patterns on the sample test strip, calibrating the light color/intensity of sample images, and creating the image light color/intensity to sample substance concentration calibration curve for a given test sample application. Many embedded programs from the manufacturers of the small computer for file organization, database management, user interface, peripheral interface, and wired/wireless communalizations, also fall into this categories. The adaptation of a general purpose small computer allows a broad range of commercial software and computer peripheral devices to be directly used.

The invented apparatus is a multiple functional device, not only in terms of the many vertical functions, such as sample substance concentration measurement, analysis, diagnosis, database and communication, also in terms of a variety of, horizontal applications, ranging from risk assessments of prenatal Down syndrome and neural tube defect fetus screening, cardiovascular disease and prostate cancer, to the monitoring of food safety, environment conditions, and illegal replica detection (through test on the quantity of key ingredients of various food, soft-drink, medicine, health, paper and chemical products), as long as medical, biomedical and chemical assay, using a test strip or micro-disc, is involved.

Different processing, analysis and diagnosis programs for given applications can be loaded onto the onboard, general purpose small computer, or data display, processing and analysis unit. These programs create a user's database for different users/patients, maintain, update, download and upload the database from and to a central computer, hold statistical data and health parameters of the general population for comparing analysis, dedicated mathematical models for risk projection and special diagnosis. The application dependent image light color/intensity to sample substance concentration calibration curve and corresponding mathematical model can be also generated using some of these programs. Application dependent user interface in different languages provides convenient use of the device as a genuine diagnosis tool. Results can be obtained from the apparatus and the programs at different stages of the operation, either the quantitative substance concentration reading, the results of comprehensive analysis, the results of the full diagnosis, or a combination of these three.

Two possible arrangements for acquiring digital images from the test sample loaded onto the invented apparatus are revealed in FIG. 5. FIG. 5(a) illustrates the set-up for a sample strips or microplate with a reflective backing, where a digital camera or the lens head of a digital camera 30; optical filters 53; an illumination (or light) source 51; an illumination enhancement and close-up lens unit 54; and the test strip or microplate 42 are involved. FIG. 5(b) illustrates the set-up for a transparent or translucent sample strip or microplate, where an illumination (or light) source 52; optical filters 53; an illumination enhancement and close-up lens unit 54; a digital camera or the lens head of a digital camera 30; and the test strip or microplate 42 are involved.

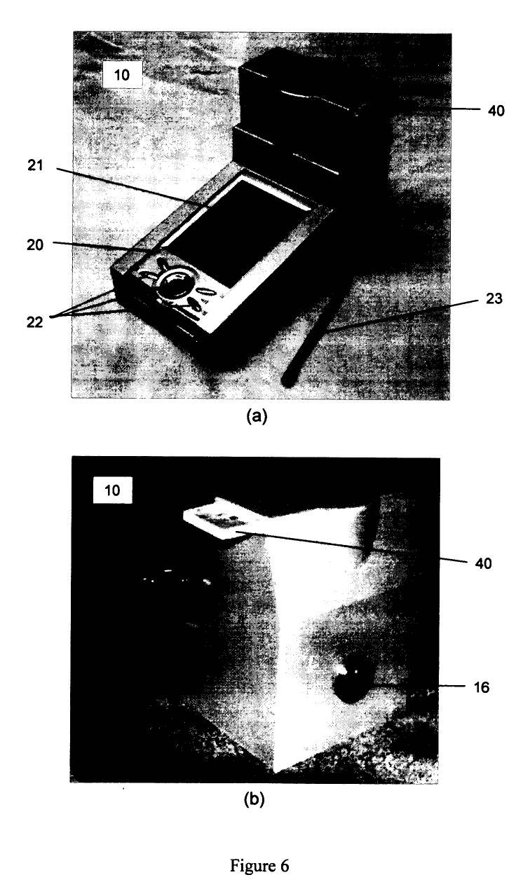

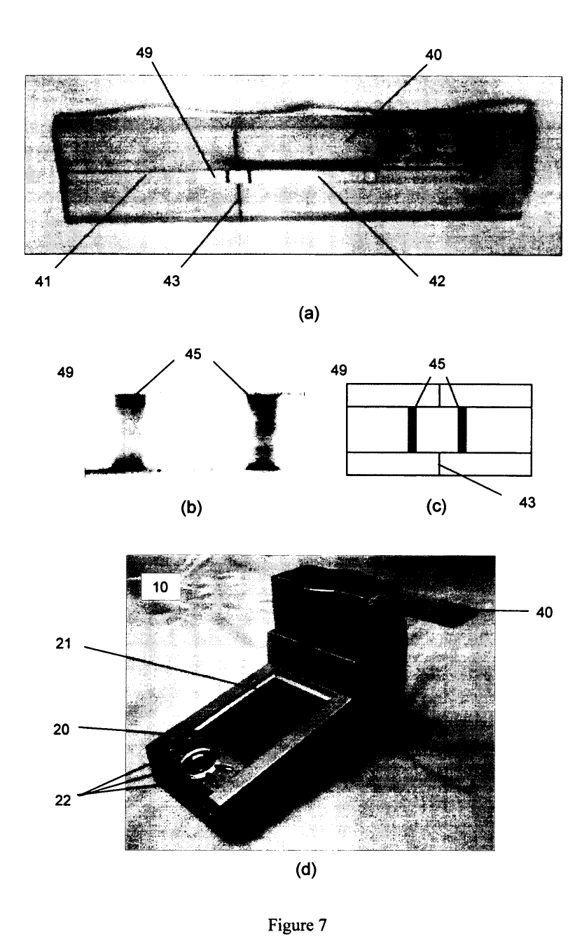

An example prototype of the invented apparatus is further illustrated using a front view of FIG. 6(a) and a back view of FIG. 6(b), where the operation screen 21, the inserted sample tray 40, the stylus of the apparatus and the onboard PDA computer 23, and the power switch of the apparatus 16 are illustrated. The insertion of the sample tray 40 is further illustrated by the photo in FIG. 7(d). FIG. 7(a) provides the details of an example sample tray 40, with a sample slot 41 fitted with a test strip 42. The reaction region of the test strip 49 is enlarged in FIG. 7(b) and its schematic view is given in FIG. 7(c). The rough alignment marker 43 and the reaction patterns 45 on the test stripe are also revealed.

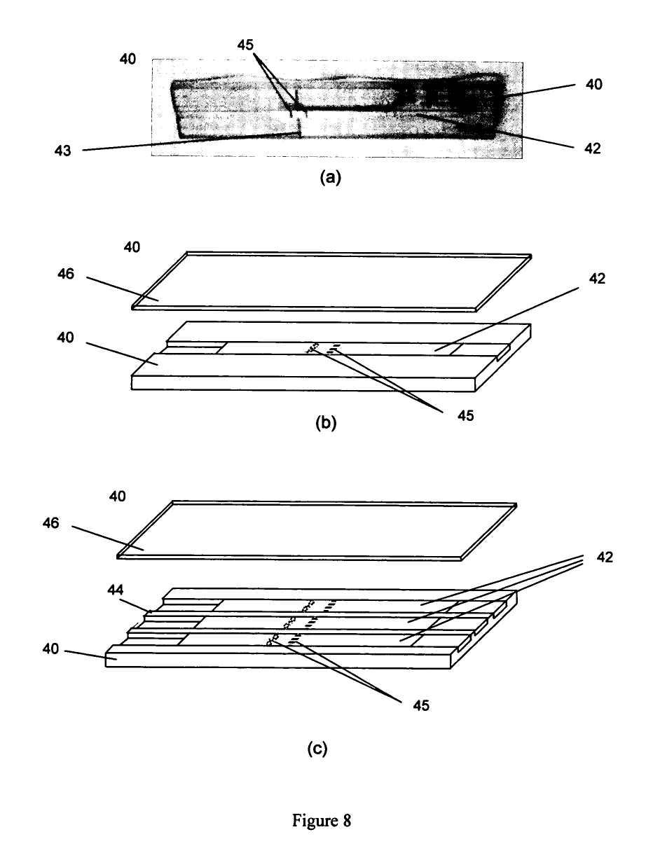

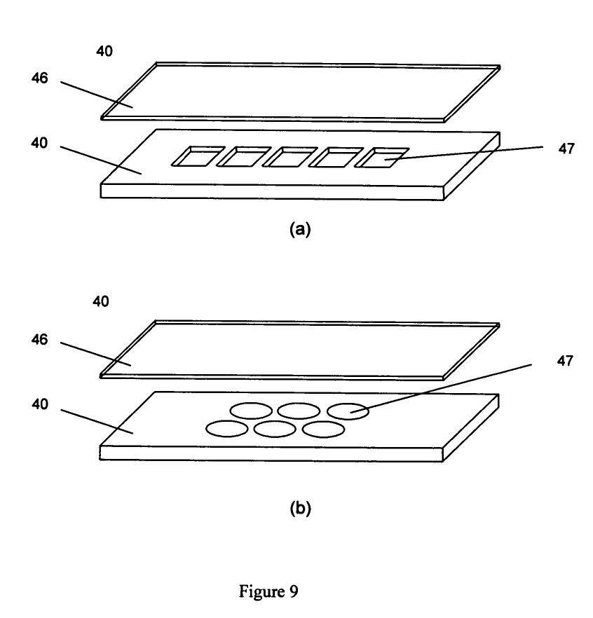

The sample tray can take different forms to accommodate a single sample or multiple samples of testing stripe, cartridge, or microplate. Possible arrangements for a single test stripe and three test stripes are illustrated in FIGS. 8(b) and (c), respectively. Sample cartridges with single and multiple arrays of microplate of square and circular shapes are illustrated in FIGS. 9(b) and (c), respectively.

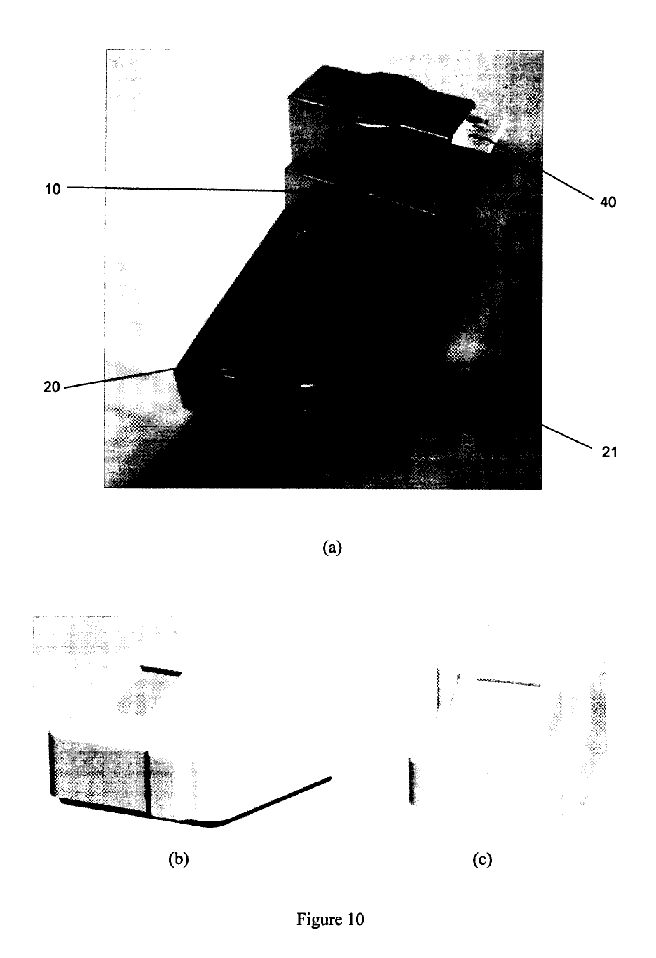

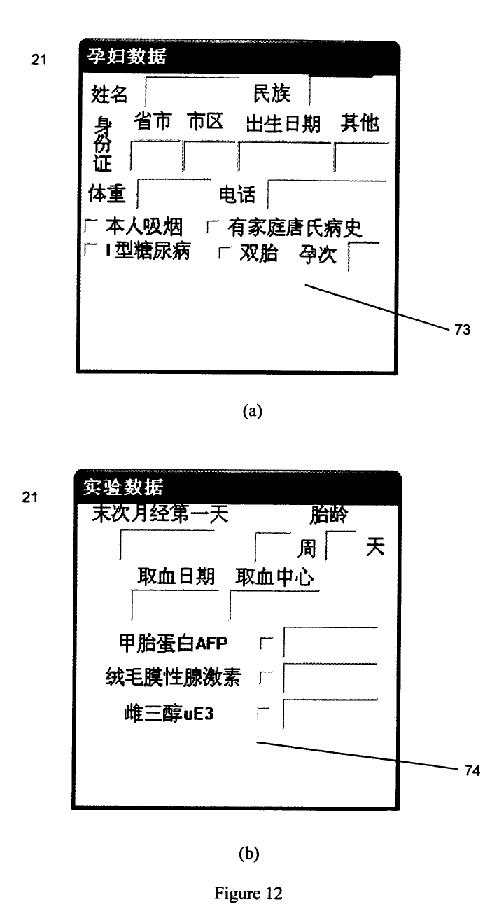

An operating example of the invented apparatus 10 is illustrated in FIG. 10(a). The acquired sample image is displayed on the touch screen 21 of the on board Personal Digital Assistant (PDA) computer 20, and the sample cartridge 40 is inserted into the cartridge slot and illuminated. Other possible implementations of the invented apparatus are illustrated in FIGS. 10(b) and 10(c), where the data display, processing and analysis unit, or computer and microprocessor, is seamlessly integrated into the apparatus. Operation of the invented apparatus normally follows the following procedures:
performing the lateral flow or microplate assay;
entering the specific information of the user or patient under examination into the computer database through the application program;
This information is used as a record of the test and diagnosis. It also provides the background information needed for carrying out the later analysis and diagnosis. Typical information includes: name, age, date of the test, body weight, etc. as illustrated in FIG. 12 through the Chinese language user interface.
putting the test strip or microplate in the sample cartridge and insert the microplate into the test sample slot;
running the image acquisition and processing program to obtain the sample reaction pattern reading, or the quantitative measurement of specific substance concentration in the sample;
carrying out analysis by associating this measurement with statistical data of the general population or average value, and performing some task specific calculation using prior knowledge and mathematical models that are built in the program; and
making specific diagnosis based on the statistical analysis, knowledge reasoning, probability and mathematical model prediction, and fizzy pattern recognition.

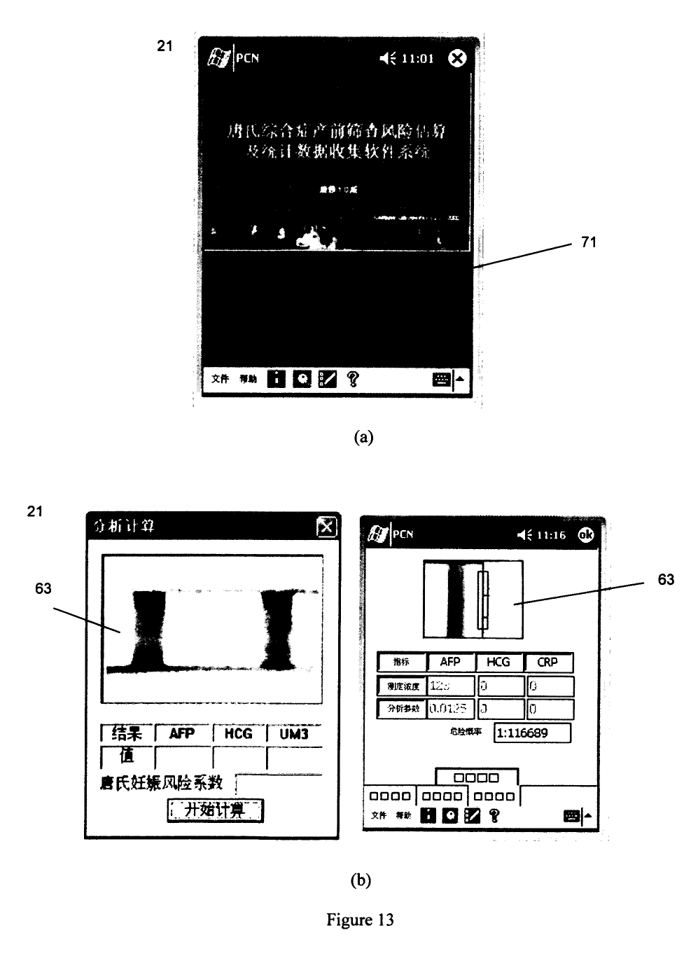

The results of the analysis and diagnosis are fed back to the user on the touch screen as shown in FIG. 13, and recorded in the database. Formal reports of the results can be automatically prepared and printed.

Prior to the stated normal operations of the invented apparatus, appropriate calibrations of the device and test strip/micro-disc are needed. This task can be carried out on the invented apparatus, or carried out for each batch of test strips and microplates at the manufacturing facility and incorporated into the invented apparatus by entering or downloading the parameters of the image light color/intensity to sample substance concentration calibration model.

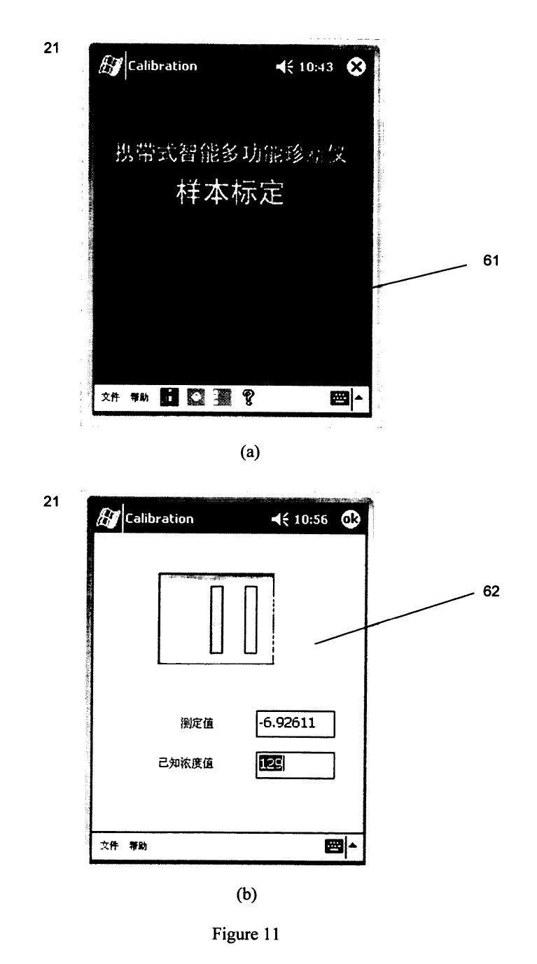

To carry out the calibration using the invented apparatus, a group of test samples with known substance concentrations covering the interested range of substance concentration variation need to be prepared. Each sample is then put into the invented apparatus in turns. The relations between the concentration of specific substances in the test samples, and the color and light intensity of the sample image are recognized, modelled and recorded by the invented apparatus, and recorded together with the user entered, known substance concentrations. These data points of the standard tests are then used to form a sample image light colour/intensity—substance concentration calibration model. Furthermore, apparatus by apparatus calibration is carried out by comparing the benchmark readings from the original and all later test images to adjust the calibration curve for each individual apparatus to give more accurate image color and light intensity readings. The parameters of this mathematical model are recorded in the database together with the batch number of the test strips and microplates. These parameters and the light colour/intensity—substance concentration calibration model defined by these parameters are kept in the apparatus to carry out the quantitative substance concentration measurements later on for the test strips and microplates of the same batch. These obtained parameters of the light colour/intensity—substance concentration calibration model can be transferred to the central computer and distributed to other apparatuses, using the same batch of test strips and microplates to support their operations. The user interface implemented in Chinese language of the light colour/intensity—substance concentration calibration program is illustrated in FIGS. 11(a) and 11(b).

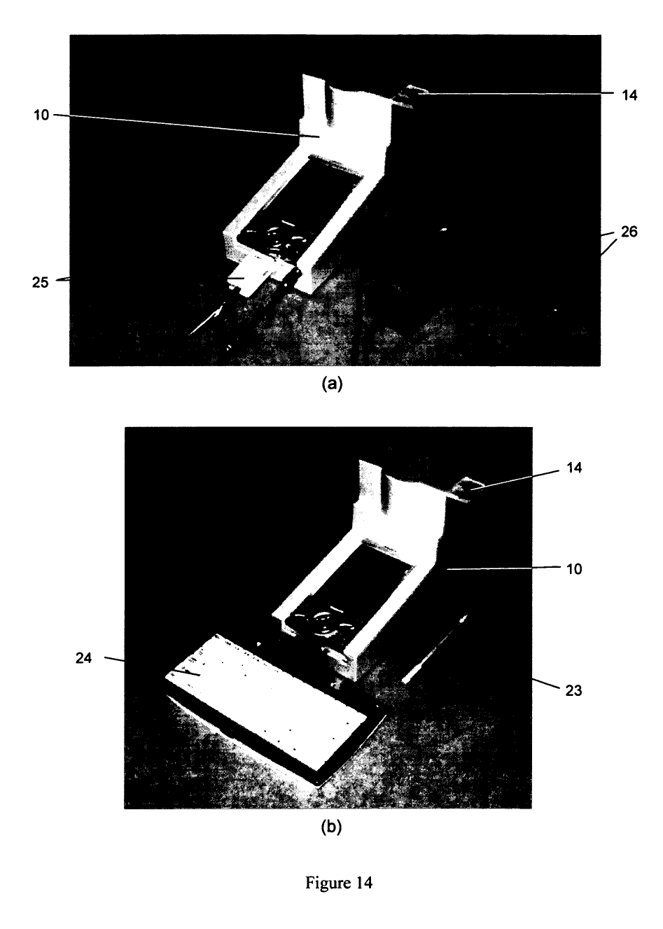

To support the operations of the invented apparatus, to record the results of the assays, analyses, and diagnoses and to pass the information to a central database, to get printed results, and to transfer model parameters, application programs and result data, a large variety of computer peripheral and communication devices are used. Additional peripheral devices that support the mobile operation of the apparatus and improve the efficiency of the apparatus are also used. In FIG. 14, a number of representative peripheral devices to the on-board computer and also to the invented apparatus, including an attachable keyboard 24; the PocketPC USB port connector 25; and the power supply connector 26, are illustrated. In most cases, the peripheral devices to a general purpose computer can be used directly as the peripheral devices of the invented apparatus.

The foregoing is a description of a preferred embodiment of the invention which is given here by way of example. The invention is not to be taken as limited to any of the specific features as described, but comprehends all such variations thereof as come within the scope of the appended claims.

1. A portable apparatus, for performing a variety of medical, biomedical and chemical assays, assay based analyses, and diagnosis, comprising
(A) a sample tray that can carry one or multiple test samples for testing;
(B) an enclosure of the apparatus that houses all components and serves as the frame;
(C) an opening on the enclosure to accommodate the sample tray that carries the test sample(s) for acquiring digital image information of the sample;
(D) an imaging acquisition system in the enclosure, which is used for acquiring digital image information of the sample;
(E) a data display, processing, and analysis unit that carries out the measurement, analysis and diagnosis on the test sample, using the acquired image information; the unit can also receive instructions, display and output results.
Among these, the enclosure and frame of the apparatus with the sample tray inserted at least contains the image acquisition system to ensure that the sample tray and the image acquisition system have a fixed relative position inside the enclosure. The image acquisition system is connected to the data display, processing, and analysis unit through digital interface, thus forming a portable device for various applications.

2. The portable apparatus of claim 1, wherein a chamber is built under the enclosure of the apparatus to hold a fixed or removable data display, processing, and analysis unit inside of the apparatus for carrying out measurement, analysis, and diagnosis. This data display, processing, and analysis unit is a general purpose computer, such as a handheld computer (HHC), a pocket personal computer (PPC), a personal digital assistant (PDA), a palm-top computer, or a laptop computer.

3. The portable apparatus of claim 1, wherein the data display, processing, and analysis unit for carrying out measurement, analysis, and diagnosis is a general purpose computer, such as a handheld computer (HHC), a pocket personal computer (PPC), a personal digital assistant (PDA), a palm-top computer, or a laptop computer. This general purpose computer can be connected to the apparatus and its image acquisition system from outside through a standard interface mounted on the enclosure of the apparatus, and removed.

4. The portable apparatus of claim 1, wherein a chamber is built under the enclosure of the apparatus to hold a fixed or removable data display, processing, and analysis unit inside of the apparatus for carrying out measurement, analysis, and diagnosis. This data display, processing, and analysis unit is a dedicated component, such as a microprocessor and associated hardware.

5. The portable apparatus of claim 1, wherein the data display, processing, and analysis unit is a dedicated component that includes a microprocessor and associated hardware. This dedicated component can be connected to the apparatus and its image acquisition system from outside through a standard interface mounted on the enclosure of the apparatus, and removed.

6. The portable apparatus of claim 1, wherein the image acquisition system hardware consists of a digital camera or the lens head of a digital camera, special illumination, and optical filters, which are all fixed to the frame and enclosure of the apparatus. The image acquisition system hardware is connected to the digital interface of the data display, processing, and analysis unit.

7. The portable apparatus of claim 1, wherein the image acquisition system hardware consists of a digital camera or the lens head of a digital camera, special illumination, and optical filters, which can be connected and disconnected to the frame and enclosure of the apparatus. The image acquisition system hardware is connected to the digital interface of the data display, processing, and analysis unit.

8. A portable apparatus, for performing a variety of medical, biomedical and chemical assays, assay based analyses, and diagnosis, comprising
(A) a sample tray that can carry one or multiple test samples for testing;
(B) an enclosure of the apparatus that houses all components and serves as the frame;
(C) an opening on the enclosure to accommodate the sample tray that carries the test sample(s) for acquiring digital image information of the sample;
(D) an imaging acquisition system in the enclosure, which is used for acquiring digital image information of the sample; the imaging acquisition system includes a digital camera or the lens head of a digital camera, special illumination, and optical filters; which are fixed onto the frame of the apparatus;
(E) a data display, processing, and analysis unit that carries out the measurement, analysis and diagnosis on the test sample, using the acquired image information; the unit can also receive instructions, display and output results.
Among these, the enclosure and frame of the apparatus with the sample tray inserted contains the image acquisition system to ensure that the sample tray and the image acquisition system have a fixed relative position inside the enclosure. The image acquisition system and the enclosure and frame of the apparatus form one body. In use, the image acquisition system is connected to the data display, processing, and analysis unit through the digital interface that is mounted on the enclosure of the apparatus, thus forming a portable device for various applications.

9. The portable apparatus of claim 8, wherein the data display, processing, and analysis unit is a general purpose computer that can be connected to the frame or enclosure of the apparatus.

10. The portable apparatus of claim 9, wherein the general purpose computer can be a handheld computer (HHC), a pocket personal computer (PPC), a personal digital assistant (PDA), a palm-top computer, or a laptop computer.

11. The portable apparatus of claim 8, wherein the data display, processing, and analysis unit is a dedicated component that includes microprocessor and associated hardware.

12. A portable apparatus, for performing a variety of medical, biomedical and chemical assays, assay based analyses, and diagnosis, comprising
(A) a sample tray that can carry one or multiple test samples for testing;
(B) an enclosure of the apparatus that houses all components and serves as the frame;
(C) an opening on the enclosure to accommodate the sample tray that carries the test sample(s) for acquiring digital image information of the sample;
(D) an imaging acquisition system in the enclosure, which is used for acquiring digital image information of the sample; the imaging acquisition system includes a digital camera or the lens head of a digital camera, special illumination, and optical filters; which are fixed onto the frame of the apparatus;
(E) a data display, processing, and analysis unit that carries out the measurement, analysis and diagnosis on the test sample, using the acquired image information; the unit can also receive instructions, display and output results. This data display, processing, and analysis unit is a dedicated component mounted on the frame of the apparatus, which consists of a general-purpose microprocessor and all associated hardware, including display screen.
Among these, the enclosure and frame of the apparatus with the sample tray inserted contains the image acquisition system and the dedicated component. Within the enclosure, the relative position between the digital camera or the lens head of the digital camera of the image acquisition system and the inserted sample tray is fixed, thus forming a portable device under the enclosure for various applications.

13. The portable apparatus of claims 1-12, wherein the data display, processing, and analysis unit includes means for operation and data entry, including control and navigator buttons, display and touch screen, USB port connector, and PocketPC USB port connection; as well as AC Adapter connector and AC power supply.

14. The portable apparatus of claims 1-12, wherein the data display, processing, and analysis unit includes stylus and on screen keyboard.

15. The portable apparatus of claims 1-12, wherein the data display, processing, and analysis unit can directly connect to various computer peripheral devices, including but not limited to the data input and output devices, speaker, microphone, infrared port, CompactFlash card slot, PC card slot, Secure Digital card slot, dock port and portable keyboard. Various wireless connections, including IrDA, GSM/GPRS, CDMA and Bluetooth, may be used.

16. The portable apparatus of claims 1-14, wherein the sample cartridge can carry one or more samples of the lateral flow or microplate assay.

17. The portable apparatus of claim 16, wherein the sample cartridge has a pocket or surface with certain width and length to hold a sample of the lateral flow or microplate assay to observe the color and light intensity of the test sample image and their changes.

18. The portable apparatus of claim 16, wherein the sample cartridge has multiple pockets or surfaces with certain width and length to hold multiple samples of the lateral flow or microplate assay to observe the color and light intensity of the sample and their changes. These samples may be separated with blank spaces.

19. The portable apparatus of claim 16, wherein the sample cartridge has a line mark to ensure the position alignments between the sample, the sample cartridge, and the enclosure of the apparatus.

20. The portable apparatus of claim 16, wherein the sample cartridge has one or multiple pockets or surfaces of circular, square, rectangular and other shapes to hold samples of microplate assay to observe the color and light intensity of the image sample and their changes.

21. The portable apparatus of claims 17-20, wherein a transparent cover is placed on top of the sample, or between the illumination light and the sample.

22. The portable apparatus of claims 1-14, wherein the image acquisition system also include filters and illumination.

23. The portable apparatus of claim 22, wherein the image acquisition system for sample strips and/or microplates with a reflective backing comprises a digital camera or the lens head of a digital camera, optical filters, an illumination (or light) source, illumination enhancement and close-up lenses, and test strips or microplates with a reflective backing.

24. The portable apparatus of claim 22, wherein the image acquisition system for sample strips and/or microplates with a transparent or transparent backing comprises a digital camera or the lens head of a digital camera, optical filters, an illumination (or light) source, illumination enhancement and close-up lenses, and test strips or microplates with a transparent or transparent backing.

25. The portable apparatus of claims 1-14, wherein a portable and plug-in power supply, preferably with a voltage stabilizer, and control device provide power to image acquisition system and the data display, processing, and analysis unit.

26. The portable apparatus of claims 1-14, wherein a regular or rechargeable battery, preferably with a voltage stabilizer, and control device provide power to image acquisition system and the data display, processing, and analysis unit.

27. The portable apparatus of claims 1-14, wherein the data display, processing, and analysis unit reads the color and light intensity of image of the test sample of a lateral flow or microplate assay using the image acquisition system, maps the sensed color and light intensity into the corresponding concentration of specific substances in the test sample, based on a stored light color/intensity to substance concentration calibration curve.

28. The portable apparatus of claim 27, wherein the measurement and analysis on the concentration of specific substances in the test samples of a lateral flow or microplate assay are based on the statistical data, prior knowledge and computer models of the subject matter, stored in the data display, processing, and analysis unit.

29. The portable apparatus of claim 28, wherein the analysis on the results of the lateral flow or microplate assay and the diagnosis from the results of the analysis are based on the quantitative measurement on the concentration of specific substances in test samples and computer analysis using the data display, processing, and analysis unit.

30. The portable apparatus of claims 1-14, wherein the data display, processing, and analysis unit carries or operates on the programs for acquiring, processing and analyzing digital images, and the programs for calibrating and recording the relations between the light color/intensity of sample image and the concentration of specific substances in the test sample.

31. The portable apparatus of claims 1-14, wherein the data display, processing, and analysis unit runs a variety of dedicated application software that are used for building and maintaining the information database of the users and/or patients; carrying out quantitative measurement of specific substances in the test sample; performing analysis based on the measured concentration of the specific substances, statistical data, prior knowledge, and mathematical models; and making diagnosis based on the results of the analysis. This data display, processing, and analysis unit in the portable apparatus also transfer the related information and the results of the analysis and diagnosis to and from the central computer and/or other portable apparatuses.

32. The portable apparatus of claims 1-14, wherein the data display, processing, and analysis unit can run a variety of application software that are required for carrying out the medical, biomedical and chemical assays, sample substance concentration measurement, analysis and diagnosis, such as the risk assessments of maternal Down and neural tube defect fetus, cardiovascular disease, and prostate cancer, and the monitoring and inspection of food and environment. These application software tools with user interface implemented in different languages can be quickly loaded on and unloaded off the portable apparatus for different applications.

33. The portable apparatus of claims 1-14, wherein the data display, processing, and analysis unit can be used to collect and update the relevant information of the sampled subjects or the users/patients, to store the information in a database, and to transfer the information to the central computers and other portable apparatus, in order to support the construction and maintenance of a computer database with both statistical and individual-specific data, such as user/patient database.

34. The portable apparatus of claims 1-14, wherein the data display, processing, and analysis unit can use various software, statistical data, users' database, sample calibration data, and computer models to process the information related to the subject sample; and can transfer these software, information and data back and forth to the central computer and/or other portable apparatuses.

35. The portable apparatus of claims 1-14, wherein the data display, processing, and analysis unit can directly use various computer software and programming environments, including but not limited to database programming and management, spread sheets, word processor, file transfer protocols, C, BAISC and other programming tools.

36. The portable apparatus of claims 1-14, wherein the data display, processing, and analysis unit can directly use various computer peripherals, including but not limited to printer, network connection, wireless communication, storage devices and attachable keyboard.

37. The portable apparatus of claims 1-14, wherein the data display, processing, and analysis unit obtains the color and light intensity of the image of the test sample from the image acquisition system of the apparatus; quantitatively measuring the concentrations of specific substances in the test sample using the stored sample image light color/intensity to sample specific substance concentration calibration curve; and automatically detects the boundaries and area of the interested color and light intensity expressing region on the sample image using image processing techniques, thus accurately detecting the color and light intensity of the interested region on the sample image and eliminating possible misalignment between the sample, sample tray and the frame of the apparatus.

38. The portable apparatus of claims 1-14, wherein the data display, processing, and analysis unit quantitatively measures the concentrations of specific substances in the sample using the color and light intensity of the acquired image of the sample, and the stored sample image light color/intensity to sample substance concentration calibration curve. The measurement uses a light color/intensity benchmark spot on the sample tray and an area on the sample adjacent to the active zone as light color and intensity background references, ensuring measurement accuracy under varying illumination, sample back material and optical filters, different apparatuses, and other changes that might be imposed by environment and production variations.

39. The portable apparatus of claims 1-14, wherein the data display, processing, and analysis unit in the apparatus quantitatively measures the concentrations of specific substances in multiple test samples of a lateral flow or microplate assay by acquiring one image, dividing the image for each sample, and processing the divided image of each sample using different algorithms.

40. The portable apparatus of claims 1-14, wherein the apparatus can be used in a method for performing and interpreting a lateral flow assay. The method includes depositing a sample on a test strip at an application region, detecting a first detection signal, in the form of changing color and/or light intensity, arising from the test strip in the first detection zone, and generating a baseline for the first measurement zone by interpolating between values of the detection signal outside of the first measurement zone and inside of the first detection zone. The method may include locating a beginning boundary and an ending boundary for the first measurement zone on the test strip. Additional detection zones having measurement zones may also be incorporated with the embodiment.

41. The portable apparatus of claims 1-14, wherein the apparatus can be used in a method for performing and interpreting a lateral flow assay includes providing a test strip on a cartridge, where the test strip includes a first analyte binding agent coupled to a detection agent and a second analyte binding agent. The method further includes depositing a sample on an application region of the test strip, where at least a portion of the sample binds to the first analyte binding agent coupled to the detection agent to form a first analyte binding agent complex, the first analyte binding agent complex moving by lateral flow to a first detection zone that includes a measurement zone, where at least a portion of the first analyte binding agent complex binds to the second analyte binding agent in the first measurement zone to form a second complex. In addition, the method also includes detecting an intensity of a first detection signal, in the form of changing color and/or light intensity, arising in the first detection zone, generating a baseline of signal intensity from the first measurement zone, and quantifying a value of signal intensity representative of the second complex with respect to the baseline.

42. The portable apparatus of claims 1-14, wherein the device can be used for:
(A) the quantitative measurement of the concentration of specific substances in test samples of a lateral flow or microplate assay and analysis;
(B) the construction and maintenance of the sample related information database of the users and/or patients, and the exchange of information with the central database on the host computer;
(C) the quantitative analysis and diagnosis based on the measured substance concentration in the test sample, stored statistical data, prior knowledge, and mathematical models.
